(19) United States
(12) Patent Application Publication
(10) Pub. No.: US 2011/0224752 A1
(43) Pub. Date: Sep. 15, 2011

(54) MICROELECTRODE STIMULATION FOR TREATMENT OF EPILEPSY OR OTHER NEUROLOGIC DISORDER

(75) Inventors: John Rolston, Atlanta, GA (US); Robert Gross, Decatur, GA (US); Steve M. Potter, Atlanta, GA (US)

(73) Assignees: Emory University, Atlanta, GA (US); Georgia Tech Research Corporation, Atlanta, GA (US)

(21) Appl. No.: 13/061,212

(22) PCT Filed: Aug. 27, 2009
(86) PCT No.: PCT/US09/55158

§ 371 Date: May 10, 2011

Related U.S. Application Data
(60) Provisional application No. 61/092,850, filed on Aug. 29, 2008.

(51) Int. Cl. A61N 1/36

(52) U.S. Cl. 607/45

(57) ABSTRACT

Methods for treating a neurologic disorder by neurostimulation. The stimulation may be applied using electromagnetic energy. In certain embodiments, distributed electrical stimulation is applied to a target site of the brain in an ongoing fashion. A microelectrode array may be used to provide the distributed electrical stimulation. The method may also comprise the detection of electrophysiologic signals from the brain. These detected signals may be analyzed and used for closed-loop feedback of the neurostimulation. Also provided are systems for neurostimulation and software for operating such systems.

### CROSS REFERENCE TO RELATED APPLICATION

The present application claims priority to U.S. provisional application Ser. No. 61/092,850, filed Aug. 29, 2008 the disclosure of which is incorporated herein by reference in its entirety.

### TECHNICAL FIELD

The present invention relates to neurostimulation for the treatment of neurologic disorders.

### BACKGROUND

Epilepsy is a debilitating, chronic illness affecting nearly one in 100 Americans and accounting for 1% of the world's burden of disease, equaling breast cancer in women and lung cancer in men. For one-third of patients, complete seizure control is unattainable, even with a combination of medications. Of these patients who are resistant to medication, surgical resection is an option for a minority, and even for those that are surgical candidates, complete control is attained in only 50-70% (depending on the location and the particular series). Long-term recurrence occurs in approximately 10% of those that do become seizure-free. Thus, there remains a very large pool of patients that require additional therapeutic options beyond medical treatment and surgical resection.

Electrical stimulation of the nervous system is a promising alternative to drug therapy and surgical resection. However, current approaches to neurostimulation for the treatment of epilepsy (e.g., vagal nerve stimulation) have had only limited efficacy. If electrical stimulation or other forms of neurostimulation are to have a greater impact on the treatment of epilepsy (and for other neurologic conditions), further improvements are needed.

### SUMMARY

In one aspect, the present invention provides a method for treating a neurologic condition in a mammalian subject, comprising: providing distributed stimulation to a site in the brain of the subject in an ongoing fashion, wherein the brain site has neurons capable of exhibiting pathologic increases in correlated neural activity. In certain embodiments, the method may further comprise positioning a microelectrode array having a plurality of microelectrodes at the brain site; and applying a stimulation signal through at least one of the microelectrodes. In certain embodiments, the method may further comprise detecting electrophysiologic signals at the brain site. The detected electrophysiologic signals may be used for providing closed-loop feedback to the stimulation.

In another aspect, the present invention provides a system comprising: a microelectrode array having a plurality of microelectrodes; and a stimulator subsystem coupled to the microelectrode array; wherein the stimulator subsystem is programmed to apply a plurality of stimulation signals to the microelectrodes in an ongoing fashion to provide distributed electrical stimulation to a site in the brain of a mammalian subject. In certain embodiments, the system may further comprise a detector subsystem for detecting electrophysiologic signals from the brain site, and wherein at least one of the microelectrodes is adapted for detecting electrophysiologic signals from the brain site.

In another aspect, the present invention provides a computer-readable storage medium having executable instructions for performing the following: receiving electrophysiologic signals from a microelectrode array; analyzing the electrophysiologic signals; using a closed-feedback loop algorithm, modifying parameters for providing distributed electrical stimulation through the microelectrode array in an ongoing fashion; and outputting a message containing commands for providing the distributed electrical stimulation.

### BRIEF DESCRIPTION OF THE DRAWINGS

FIGS. 1A-1C are schematic illustrations depicting a possible physiologic mechanism of action by which distributed stimulation may work to suppress the burst firing of neurons. FIG. 1A shows a set of neurons receiving afferent input from another part of the brain. FIG. 1B shows the neurons having the afferent input cut-off. FIG. 1C shows a microelectrode array providing electrical stimulation to the neurons.

FIG. 2 shows a block diagram of a system according to an embodiment of the present invention.

FIGS. 3A and 3B shows various stimulation signal waveforms that may be applied by certain embodiments of the present invention.

FIG. 4 shows representative tracings of action potentials and local field potentials that may be recorded by certain embodiments of the present invention.

FIGS. 5A and 5B show representative power spectrums that may be obtained from recordings made by certain embodiments of the present invention.

FIG. 6 shows a block diagram of a system according to another embodiment of the present invention.

FIG. 7 shows microelectrode recordings of evoked action potentials before and after a stimulation pulse at varying voltage levels.

### DETAILED DESCRIPTION

The present invention relates to neurostimulation for the treatment of various neurologic disorders including epilepsy. In one aspect, the present invention provides a method for treating a neurologic disorder in a mammalian subject, such as a human patient. The method comprises applying distributed stimulation at a target site of the subject's brain. The target site may be a site where neurons are capable of exhibiting pathologic increases in correlated neural activity, such as, for example, pathologic bursting or burstiness, synchronized firings, oscillatory firings, or pulsating activities. Such areas of the brain include, for example, the limbic system, amygdala, hippocampus (e.g., CA1, CA2, or CA3), entorhinal cortex, dentate gyms, subthalamic nucleus, globus pallidus, thalamus, striatum, and others.

As used herein, the term “distributed,” when referring to the stimulation, means that the stimulation is applied to the target site at two or more discrete, spatially separated locations (as opposed, for example, macrostimulation provided using an electrode and counter-electrode). The temporal characteristics, temporal relationships, and spatial characteristics of the distributed electrical stimulation can vary depending upon the particular application. Individual locations or groups of different locations may be stimulated simultaneously or non-simultaneously with respect to each other. Where the individual locations or groups of different locations are stimulated non-simultaneously, the sequence of stimulation may be random (including pseudorandom sequences) or be pre-determined.

The stimulation is applied in an ongoing fashion. As used herein, the term “ongoing,” when referring to the stimulation, means that the on-time of the stimulation is not dependent upon the detection of abnormal neural electrophysiologic activity (e.g., epileptiform patterns on electroencephalogram) in the subject. As such, the stimulation may occur whether or not there is abnormal neural electrophysiologic activity occurring in the subject.

For example, in the case of epilepsy or other seizure disorder, the stimulation would occur whether or not the subject's electrophysiologic activity is indicative of a normal, baseline, pre-ictal, ictal, or post-ictal stage. This does not mean that the present invention requires the measurement of electrophysiologic activity in the subject—the present invention may or may not be practiced with the detection of neural electrophysiologic activity. In some cases, where the neurologic condition is epilepsy, the stimulation is provided for a duration encompassing at least a portion of an interictal period in the subject; and in some cases, for a duration encompassing an entire interictal period in the subject.

Also, the term “ongoing” does not mean that the stimulation is necessarily unending or without interruptions. For example, interruptions may occur incidentally during the operation of the system, with system resets or updates, or while clinical procedures are being performed on the subject. Furthermore, the term “ongoing” does not necessarily mean that the signal used in the stimulation must be continuous. For example, the stimulation signal may be pulsatile or have waveform characteristics (e.g., sinusoidal).

In vitro studies of mammalian cortical cultures have demonstrated that a prominent feature of the electrical activity of high-density dissociated cortical cultures is their propensity for synchronized bursting. Bursting is also known to occur in vivo and is thought to be involved various neurologic pathology, including epilepsy. Some experts believe that burst firing of cortical output neurons may be the final common pathway for the expression of clinical seizure activity. As such, by suppressing the burst firing of neurons, improved seizure control may be achieved.

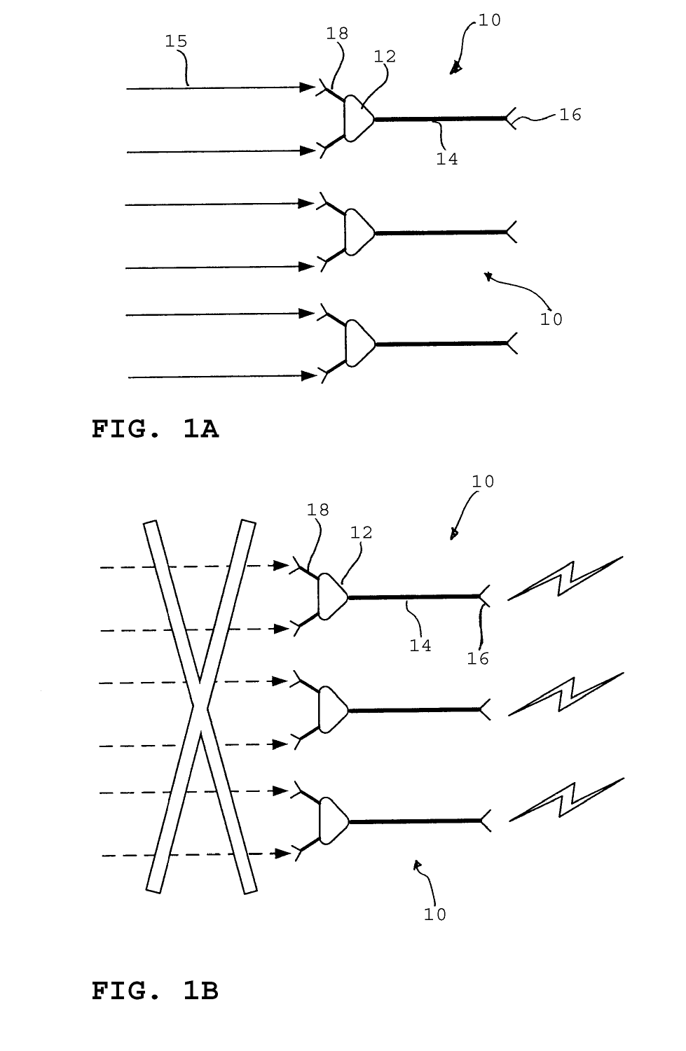

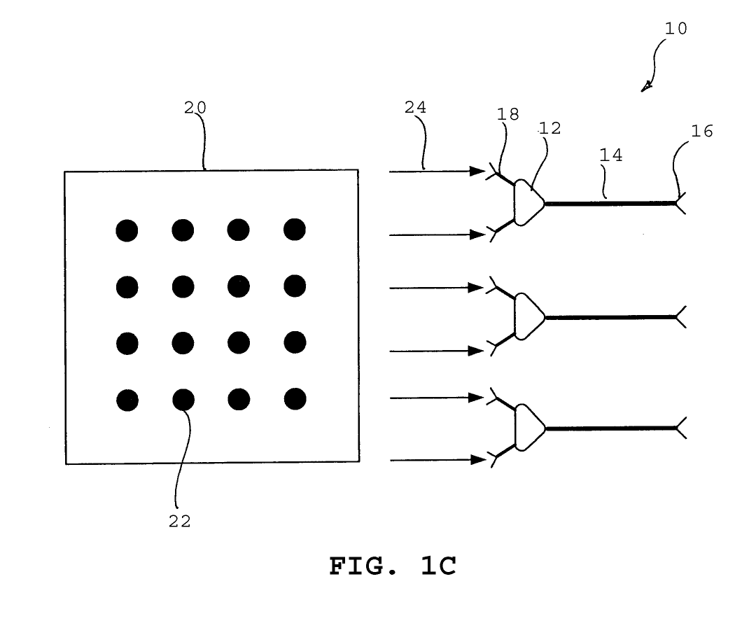

Without intending for the present invention to be bound by theory, FIGS. 1A-1C show a possible physiologic mechanism of action by which the distributed stimulation may work to suppress the burst firing of neurons. FIG. 1A shows a set of neurons 10 having cell bodies 12, axons 14, axon terminals 16, and dendrites 18. At dendrites 18, neurons 10 receive afferent input 15 from other parts of the brain (e.g., cortical or subcortical structures), which can serve to inhibit the activity of neurons 10.

The persistence of bursting in dissociated cortical cultures is believed to be a result of deafferentation of neurons. It is known that deafferentation can play a role in epilepsy, where the epileptic focus is cut-off from the afferent drive of the subcortical, entorhinal, or other limbic input. This scenario is depicted in FIG. 1B, which shows the deafferentation of neurons 10 with the cutting-off of afferent input 15. Without afferent input 15, neurons 10 may exhibit persistent bursting behavior. FIG. 1C shows how the use of a microelectrode array 20 may operate to suppress the bursting behavior of neurons 10. The distributed, ongoing stimulation 24 provided by the microelectrodes 22 of microelectrode array 20 may serve as a substitute for the absent afferent communication to neurons 10, thus causing neurons 10 to become “reafferented.” By maintaining neurons 10 in a state of tonic, non-bursting action potential activity, bursting activity or other type of pathologic increases in correlated neural activity may be suppressed.

The stimulation may be applied using any type of electromagnetic energy suitable for stimulating neural tissue. Such suitable types of electromagnetic energy include, for example, electrical, optical, magnetic, or radiofrequency (RF) energy. In certain embodiments, the present invention is implemented using electrical stimulation. The distributed electrical stimulation may be provided by any suitable electrode device. In certain embodiments, the distributed electrical stimulation is provided by a microelectrode array positioned at the target site of the brain. The microelectrode array has multiple microelectrodes capable of delivering highly localized electrical stimulation at the target site. The stimulation signal is applied through at least one of the microelectrodes of the microelectrode array. Where the stimulation signal is applied through multiple (i.e., two or more) microelectrodes, the stimulation signal through each of the microelectrodes may be controlled independently. In some cases, the microelectrodes may also be capable of recording with each microelectrode of the microelectrode array being dedicated to stimulation or detection, or being switchable between detection and stimulation functions.

The microelectrode array may be any of those known in the art that are suitable for use in brain stimulation. The design characteristics of the microelectrode array will vary depending upon the needs of the particular application, including such features as the number of microelectrodes, number of independent channels, spacing of the microelectrodes, positioning or arrangement of the microelectrodes (e.g., a grid arrangement or other type of multi-contact arrangement), contact area or size of the microelectrodes, geometry of the microelectrodes, and the configuration of the microelectrode array (e.g., planar, cylindrical, annular, square, etc.).

For example, the dimensions of the microelectrodes can vary depending upon the spatial precision needed for the particular application. In some cases, the microelectrodes may have a dimension of less than 1 mm as measured along its longest axis, a diameter of 10-100 μm, and/or a contact area in the range of 100-10,000 μm², which can be suitable for recording signals from single or multi-unit neuronal activity. Such microelectrodes can have impedance levels sufficient for the precise measurement of action potentials from single or multi-unit neural activity (e.g., 0.1 MΩ or higher). Also, for such applications, the spacing between microelectrodes may be in the range of 50 to 1000 μm.

The electrical stimulation being applied may be characterized according to various parameters, including voltage, current amplitude, pulse width, frequency (e.g., the stimulation rate of electrodes individually or array-wide), train length, or waveform. Such stimulation parameters will vary depending upon the particular application and can be selected according to various considerations, including the magnitude of the neurologic events being experienced by the subject, the magnitude of the stimulation necessary to modulate the neurons, or the characteristics of the microelectrodes. For example, the voltage may be selected from a range of ±0.1-10 V, pulse width may be selected from a range of 50-400 μs per phase, array-wide stimulation frequency may be selected from a range of 50-200 Hz, individual electrode stimulation frequency may be selected from a range of 0.5-200 Hz, and current may be selected from a range of ±0.1-100 μA.

The stimulation signal can have any suitable waveform, including square, sinusoidal, sawtooth, or spiked, and where applicable, the signal may be monophasic, biphasic, multiphasic, or asymmetric. In some cases, where the microelectrodes are also used for recording in addition to stimulation, the pulse width may be constrained by the need for a recording window between the pulses. Furthermore, the stimulation may be voltage-controlled or current-controlled, depending upon various considerations, such as circuit complexity, potential harm to tissue, or potential damage to the microelectrodes. In some cases, the stimulation may be current-controlled. This feature can be useful because the electric field and potential near the electrodes can be directly calculated and the effects of current-controlled stimulation are thought to be better understood that the effects of voltage-controlled stimulation.

In some cases, the stimulation parameters can be selected empirically based on baseline activity or previous performance of the neurostimulation system. For example, in some cases, negative pulses may be effective in evoking responses while positive pulses are not. Also, for example, biphasic pulses (positive phase first) may be more effective than monophasic negative pulses. In some cases, a low frequency (1-50 Hz array-wide), low current amplitude (0.1-30 μA) stimulation signal may be used. Stimulation signals having current amplitudes within this range can provide current densities suitable for evoking responses from neurons.

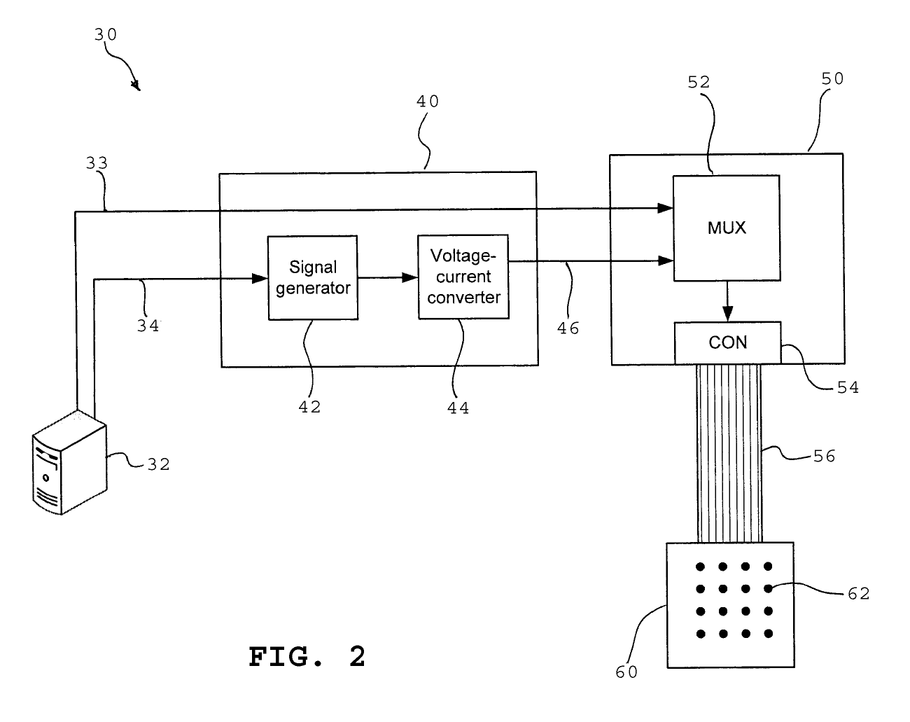

Referring to the example embodiment shown in FIG. 2, a neurostimulation system 30 includes a general purpose computer 32, an interface module 40, a headstage 50, and a microelectrode array 60. Computer 32 can serve various functions in system 30, including providing a user interface, sending commands and data for generating neurostimulation signals, performing on-line or off-line analysis, or storing a knowledge base used in the implementation of the present invention. Computer 32 is coupled to interface module 40 via control line 34. As used herein, the terms “coupled” or “coupling” refers to a signaling relationship between the components in question, including direct connection or contact (e.g., via an electrically or optically conductive path), radio frequency (RF), infrared (IR), capacitive coupling, and inductive coupling, to name a few.

Interface module 40 is adapted to generate the signals used for the electrical stimulation being applied to the target site in the subject. This can be accomplished using any of a number of different circuit configurations and components. In this particular embodiment, a signal generator 42 in interface module 40 receives messages containing commands and/or data from computer 32. Based on the commands and/or data received from computer 32, signal generator 42 generates electrical stimulation signals having the desired characteristics (e.g., waveform, pulse width, frequency, etc.). The output of signal generator 42 is passed through a voltage-to-current converter 44 to provide for current-controlled stimulation.

Interface module 40 is coupled to a headstage 50, which is mounted on the subject's head. Via a communication line 46, the stimulation signals generated by interface module 40 are fed into a multiplexer 52, which is controllable (via control line 33 from computer 32) to route the stimulation signal to the appropriate channel in a properly time-correlated manner. Via a multi-channel cable 56 connected to connector 54, the multiple outputs of multiplexer 52 are transmitted to the individual microelectrodes 62 of microelectrode array 60 through its assigned channel. As such, the electrical stimulation signal provided to each of microelectrodes 62 can be controlled independently to provide distributed electrical stimulation in the manner desired (e.g., sequentially, substantially simultaneously, or in groups).

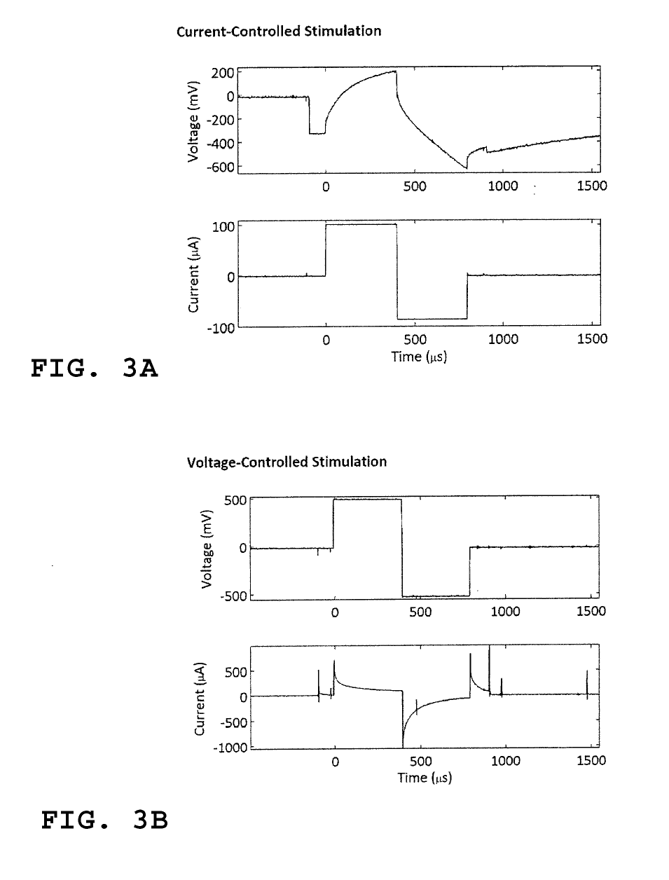

FIGS. 3A and 3B show representative pulse shapes that can be applied by stimulation system 30. In an example of current-controlled stimulation, FIG. 3A shows the voltage applied to generate a biphasic (positive phase first) rectangular-shaped current pulse being. In an example of voltage-controlled stimulation, FIG. 3B shows a biphasic (positive phase first) rectangular-shaped voltage pulse being applied and the resulting current waveform.

In certain embodiments, the stimulation is applied using optical energy, which can be, for example, red or infrared light. The optical stimulation may be provided using any suitable device capable of delivering highly localized optical energy to the target site. For example, such devices may employ optical fibers, lasers, and/or light-emitting diodes (LEDs) for the delivery of optical energy to the target site. One such device is disclosed in U.S. Patent Application Publication No. 2007/0060984 (Webb et al.), which is incorporated by reference herein. The optical energy is provided at wavelengths and intensities suitable for evoking action potential responses in the neurons. In some cases, the neurons at the target site have light-activated ion channels, such as, for example, channelrhodopsin-2 (ChR-2) or other light-activated 7-transmembrane ion channels. Neurons having such light-activated ion channels may exhibit enhanced response to optical energy. To cause expression of the light-activated ion channels, the neurons at the target site may be treated or have been treated by transfection of the appropriate genetic material (e.g., by using a viral vector containing DNA encoding ChR-2).

In certain embodiments, the present invention further comprises the monitoring of neural activity by the detection of electrophysiologic signals from the brain, which may be performed simultaneously or continuously with the stimulation. The electrophysiologic signals may be detected from the same target site as the stimulation or at a different site. Where a microelectrode array is used, activity can be measured from one or more of the microelectrodes. In some cases, it may be desirable to record from several regions of the target site in order to better characterize its activity. The terms “record” and “detect,” when referring to the electrophysiologic signals, are intended to be used interchangeably herein.

Various types of electrophysiologic signals from various sources or populations of neurons may be detected. For example, single unit or multi-unit neuronal activity can be detected using microelectrode recordings. For example, action potentials are believed to originate from cell bodies or axons located within 100 μm of the microelectrode tip; local field potentials (LFPs) are believed to represent the summed synaptic input of thousands of neurons within a roughly 1 mm³ volume of neural tissue; and epileptic spikes on electroencephalogram (EEG) are believed to represent the summations of excitatory post-synaptic potentials (EPSPs) of many dendrites.

The different types of electrophysiologic signals may be extracted for analysis. Extraction of the desired signals may be performed by any suitable signal processing technique, including analog or digital filtering. For example, single or multi-unit action potentials may be extracted by high-pass filtering (e.g., 500-9,000 Hz) of the electrophysiologic signals. LFPs may be extracted by low-pass filtering (e.g., 1-500 Hz) of the electrophysiologic signals. Action potentials and LFPs may also be extracted from other frequency bands. Furthermore, other types of signals may be extracted from other frequency bands (e.g., pathologic oscillations, high frequency oscillations in the range of 80-600 Hz, ripple oscillations in the range of 100-200 Hz, or fast ripple oscillations in the range of 250-600 Hz). Further processing of the signal may include feature extraction (e.g., spike detection) or the suppression of noise or stimulation artifacts.

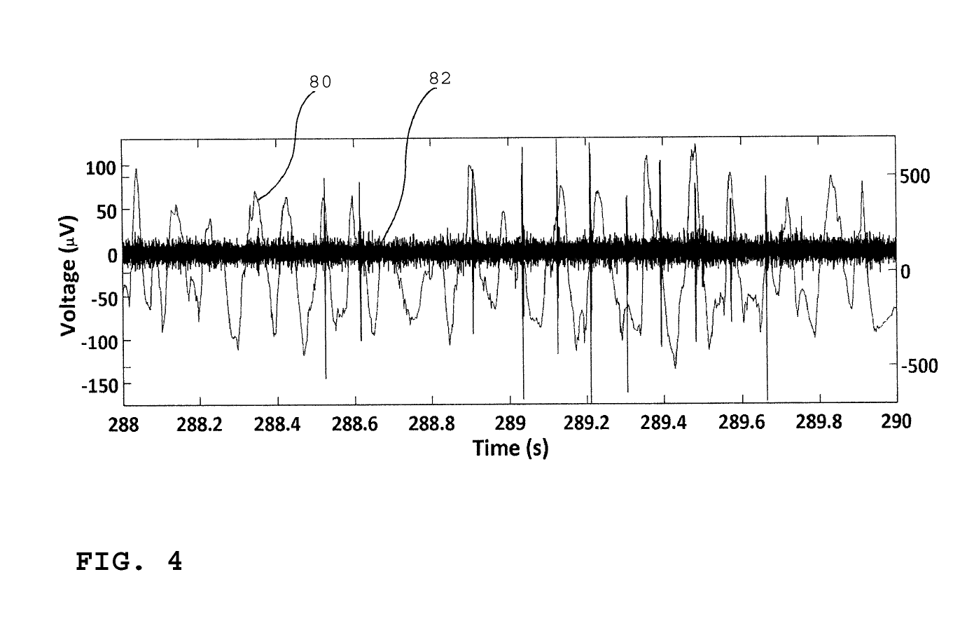

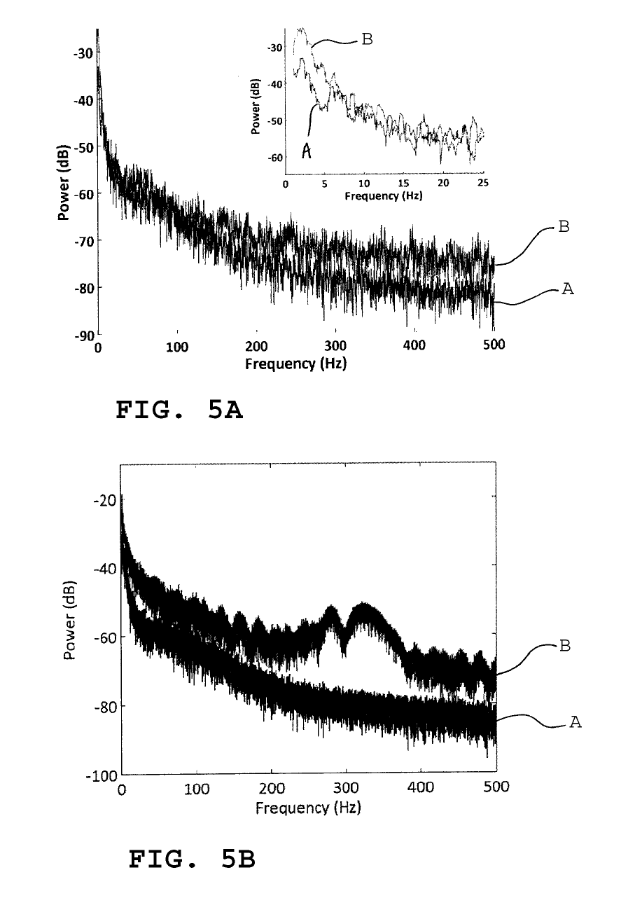

The tracings shown in FIG. 4 is a representative example of the types of signals that can be obtained by appropriate filtering. The tracings were obtained from a microelectrode implanted in the hippocampus of a normal, awake, adult rat. Tracing 82 represents action potentials obtained by high-pass filtering at 300 to 9,000 Hz, and tracing 80 represents LFPs obtained by low-pass filtering at 1 to 300 Hz.

The detected electrophysiologic signals may be analyzed using any suitable signal analysis technique known to be used for characterizing neural activity. In some cases, the signal analysis involves the dynamic extraction of neuron firing features. For example, high-pass filtered signals may be analyzed to quantify the firing rate of the neurons. When the firing rate of neurons are detected after a stimulation pulse, it may be desirable to identify the action potentials that are time-locked to the stimulus with latencies characteristic of the local neural network (e.g., <200 msec) around the microelectrode that delivers the stimulus.

In another example, the electrophysiologic signals may be analyzed to quantify the level of bursting by taking into account the number of bursts and the size of the bursts (e.g., the number of participating neurons, the aggregate number of action potential spikes, or duration). For example, the following calculations may be used in the construction of a histogram. Divide a 5-minute array-wide recording of spikes into 300 one-second long time bins. Count the number of spikes in each bin. Compute the fraction Φ₁₅ of the total number of spikes in the 5-minute recording that is accounted for by the top 15% of bins with the highest number of counts. If the firing rate of the neurons is tonic (indicating a low level of bursting), Φ₁₅ will be close to 0.15. However, if there is a high level of bursting such that most of the spikes are contained in bursts, Φ₁₅ will be close to 1 if the bursts do not occupy more than 45 bins (i.e., 15% of the one-second long bins) in a 5-minute recording. A burstiness index (BI) normalized between 0 (no bursts) and 1 (burst dominated) can be defined as:

$$BI = \frac{{Φ}_{15} - 0.15}{0.85}$$

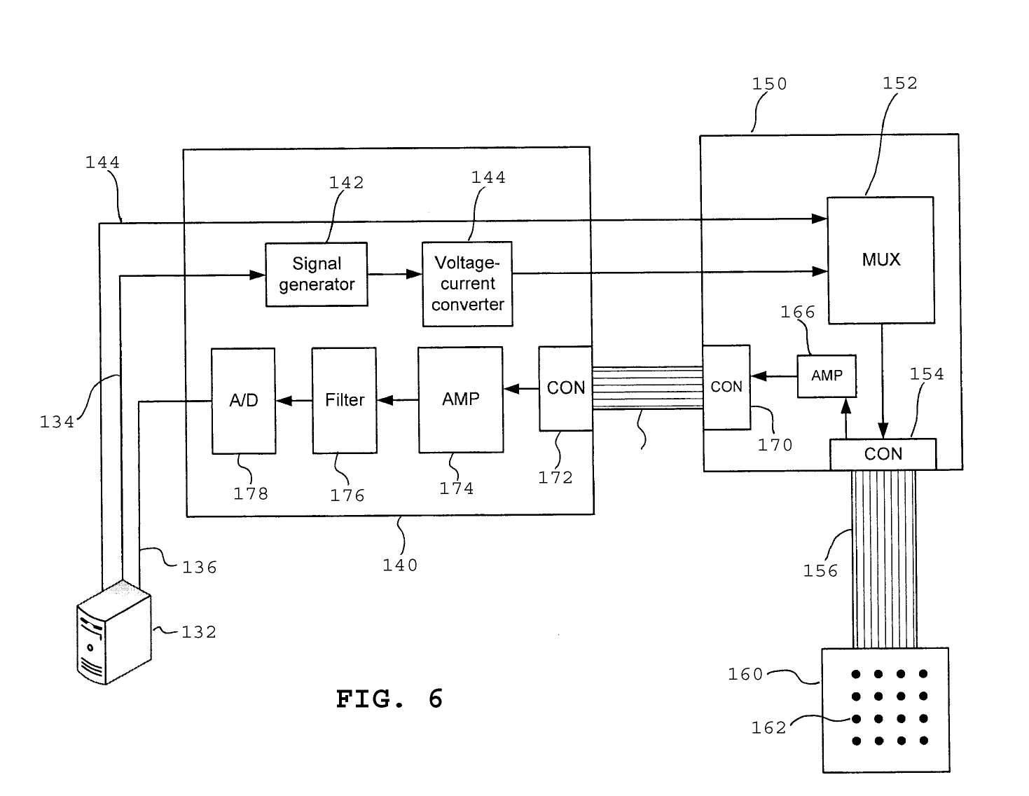

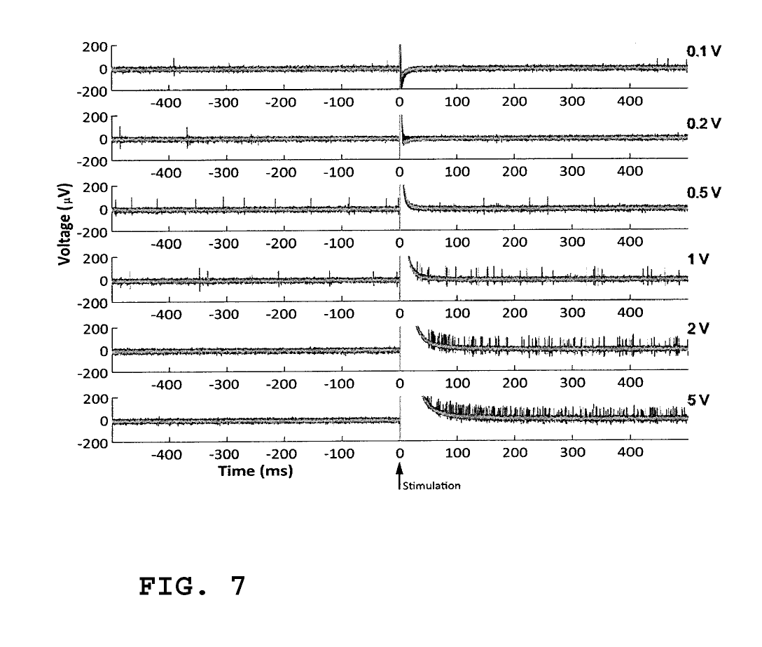

In another example, the power spectrum or spectral derivatives of the detected electrophysiologic signals may be determined and analyzed. Spectral analysis can be performed using any conventional technique, including the use of commercially available software that use multi-taper methods. The graphs shown in FIGS. 5A and 5B are representative examples of power spectra that can be obtained from microelectrode recordings in a mammal. In FIG. 5A, plot A represent the power spectrum of low-pass filtered signals from the neocortex of neurologically normal rats. Plot B represents the power spectrum from the neocortex after tetanus toxin injection into the neocortex of the rats, causing the rats to become epileptic. Similarly, in FIG. 5B, plot A represent the power spectrum of low-pass filtered signals from the hippocampus of neurologically normal rats. Plot B represents the power spectrum from the hippocampus after tetanus toxin injection into the hippocampus of the rats, causing the rats to become epileptic. These results demonstrate that neural activity in the brain of mammals can be characterized by power spectrum analysis.

Neural activity can also be characterized by statistical analysis of the detected electrophysiologic signals. For example, referring back to FIG. 4, the relationship (e.g., cross-correlation) between the spikes and local field potentials (spike-field coherence) may be analyzed. One way of calculating coherence between the two signals is by calculating the covariance C(x,y) given by the cross-spectrum normalized by the square root of the product of the individual autospectra for the signals, using the following function:

$$Cxy ( f ) = \frac{Sxy ( f )}{\sqrt{\vert Sxx ( f ) \vert \vert Syy ( f ) \vert}}$$

In some cases, the detected electrophysiologic signals may be used in a closed-loop feedback algorithm for modifying the stimulation. In some cases, the closed-loop feedback system operates in real-time. As used herein, the term “real-time” means a delay in the analysis and adjustments that is of sufficiently short duration to provide feedback at biologically relevant time-scales (e.g., corresponding to a few typical neuron-to-neuron propagation delays). In some cases, the delay may be less than 500 msec; and in some cases, less than 5 msec. The delay may be continuous or persistent, but is non-cumulative.

In many cases, it is desirable to apply stimulation energy at intensity levels that minimize the amount of harm caused to neural tissue (e.g., destructive ablation). By using a closed-loop feedback system, a lower stimulation intensity may be needed to reliably maintain a tonic firing rate in the stimulated neurons. As such, in the case of electrical stimulation using a microelectrode array, it is believed that fewer microelectrodes, lower voltage, and/or less current may be needed. Furthermore, by using a closed-loop feedback system, it is believed that habituation or long-term effects of chronic stimulation can be avoided and/or that power consumption can be reduced, which can reduce battery requirements.

The feedback algorithm may make adjustments to the stimulation parameters, which may be any of those described above. The feedback algorithm may adjust the stimulation parameters as a function of one or more of the measures of neuronal activity, including those described above. For example, the closed-loop system may monitor neuron firing rates (e.g., array-wide firing rates) or the bursting of neurons and make rapid, real-time adjustments in the stimulation parameters to increase the reliability of neuron firing responses at the desired level or to reduce bursting levels.

Referring to the example embodiment shown in FIG. 5, a stimulation system 130 includes components that are similar to those described in stimulation system 30 shown in FIG. 2, including a general purpose computer 132, an interface module 140, a headstage 150, and a microelectrode array 160. Interface module 140 contains a signal generator 142 and voltage-to-current converter 144 to generate the signals used for the electrical stimulation being applied to the target site in the subject. Headstage 150 includes a multiplexer 152 for routing of the signals to the appropriate channels of microelectrode array 160. The signals are transmitted through connector 154 and multi-channel cable 156 to the selected microelectrodes 162 of microelectrode array 160.

Each of the microelectrodes 162 of microelectrode array 160 are also capable of recording electrophysiologic signals at the level of single or multi-unit action potentials, with rapid switching between stimulation and recording functions. As such, the application of a stimulation signal to the target site and the detection of electrophysiologic signals from the target site may be performed through the same microelectrode in an alternating fashion. Between stimulation pulses, the signals recorded from each of microelectrodes 162 are transmitted individually through multi-channel cable 156 to connector 154. The recorded signals are impedance-matched and pre-amplified to line level by amplifier 166, sent through connector 170, and then transmitted to interface module 140 through a multi-channel cable 158 that connects interface module 140 to headstage 150. At interface module 140, the signals are fed into an amplifier 174 and the output from amplifier 174 is then passed through a bandpass filter circuit 176. Digital conversion of the bandpass filtered signals is performed by an analog-to-digital converter 178, and via data line 136, the digitized output is sent to computer 132 for further processing. The recorded signals may be sampled at rates sufficient for the particular data analysis needs. For example, sampling at 25 kHz can be sufficient for the analysis of action potential spikes and sampling at 2 kHz can be sufficient for the analysis of local field potentials. Using any suitable spike detection algorithm, computer 132 determines the number of action potential spikes detected array-wide.

In this particular embodiment, an initial array-wide target firing rate is set at 5 times the array-wide spontaneous firing rate. In a continuous manner, closed-loop feedback is used to adjust the stimulation parameter(s) as a function of the detected array-wide firing rate of the neurons. For example, where system 130 applies voltage-controlled stimulation, the amount of voltage applied may be adjusted as a function of the number of action potentials detected array-wide per 500 ms window. One such function could be represented as follows:

$$V ← V [ 1 - ɛ ( \frac{f - {f}_{0}}{{f}_{0}} ) ]$$

where the variable f is the number of action potentials detected array-wide in the previous 500 ms window, ƒf₀ is the target firing rate, and ε is a gain factor determining how rapidly V is adjusted according to changes in the detected array-wide firing rate. The gain factor ε may be selected to provide rapid feedback and preventing oscillations caused by overcompensation. For example, where V is updated every 100 msec, ε may be set to 0.02, corresponding to a time constant of 5 seconds.

It is possible that the stimulation efficacy may vary between the microelectrodes in the microelectrode array, depending on various factors such as the unique characteristics of each microelectrode or the characteristics of the neurons at the microelectrode tip. To account for such performance variations, the detected firing rate at each individual microelectrode may be measured and used to fine-tune the stimulation signal being applied to the individual microelectrodes. For example, for each individual microelectrode k, a running average fₖ of the detected firing rates may be maintained. The average can be determined within a moving window of the 20 most recent stimuli. A fine-tuning factor αₖ is set as:

$${α}_{k} = \frac{η}{{f}_{k}}$$

wherein η is a normalization factor to make 1 the average of all αₖ values. For the next stimulus on microelectrode k, the voltage is set at:

Vₖ=αₖV

In some cases, the feedback algorithm may use neural activity measured at a site remote from the stimulation target site. For example, stimulation may be applied to CA3, while receiving feedback from CA1. In another example, stimulation may be applied to a subcortical nuclei (e.g., the subthalamic nuclei), while receiving feedback from a neocortical epileptic focus.

The data obtained from the recordings may be further analyzed off-line by computer 132. Off-line analysis can be used for various purposes, including re-tuning of the adaptive parameters, setpoint readjustments, or as a research tool for obtaining a better understanding of the physiologic mechanisms that operate during epileptic seizures or other disturbances, gathering information from different subjects to form a database for research and development, and investigating new algorithms or features for controlling bursting.

In other aspects, the present invention provides a system that is programmed to perform the functions and capabilities as described above. Various functions and capabilities of the systems disclosed herein (e.g., the controller) may be performed by electronic hardware, computer software (or firmware), or a combination of both. Further, the division of work between the functional subsystems can also vary. For example, in the system of FIG. 6, the work involved in the filtering and processing of the detected signals may be divided between interface module 140 and computer 132. Furthermore, the functional distinctions illustrated in FIG. 6 may be integrated in various ways. For example, all the signal processing components on interface module 140 may be integrated into a single digital signal processor (DSP). Thus, while the block diagram of FIG. 6 makes functional distinctions for the sake of clarity and understanding, there may not be meaningful distinctions in an implementation of the present invention. Furthermore, although in FIG. 6, interface module 140 and headstage 150 are contained in separate physical enclosures, in other embodiments, all the capabilities described above may be contained in a single physical enclosure, or a plurality of separate physical units may perform subsets of the above-described capabilities.

Also, the various systems described herein may each include a computer-readable storage medium having executable instructions for performing the various processes as described and illustrated herein. The storage medium may be any type of computer-readable medium (i.e., one capable of being read by a computer), such as hard drive memory, flash memory, floppy disk memory, optically-encoded memory (e.g., a compact disk, DVD-ROM, DVD±R, CD-ROM, CD±R, holographic disk), or a thermomechanical memory (e.g., scanning-probe-based data-storage). The systems disclosed herein may also include addressable memory (e.g., random access memory or cache memory) to store data and/or sets of instructions that may be included within, or be generated by, the executable instructions when they are executed by a processor on the respective platform. For example, a computer used in a system of the present invention may have executable instructions for analysis of the detected electrophysiologic signals and modifying the stimulation signals based on a closed-loop feedback algorithm.

The present invention can be used for treating any of various types of neurologic disorders that are characterized by aberrant oscillating, bursting, or pulsating activity. Such neurologic diseases include epilepsy and other seizure disorders, movement disorders involving the basal ganglia (e.g., Parkinson's disease), essential tremor, multiple sclerosis, chronic pain (including neuropathic pain), headache, tinnitus, Tourette's syndrome, drug addiction, eating disorders, schizophrenia, depression, anxiety, or obsessive-compulsive disorder.

Experimental studies were conducted on male Sprague-Dawley rats weighing 35030 grams. The rats were anesthetized with isoflurane and a large rectangular craniotomy was opened with a high-speed dental drill. A microelectrode array (16-channel×50 μm diameter polyimide-insulated tungsten microelectrodes; Tucker-Davis Technologies) was implanted in the motor cortex or hippocampus (CA3 and CA1) of the rats, and the craniotomy was then sealed.

Neural activity from the rats was buffered and impedance-matched through a recording headstage mounted on the animal's headcap. The signals were passed through a bandpass filter that separated the recorded signals into two streams: 0.7-500 Hz for local field potentials and 300-8800 Hz for action potentials. The signals were fed through a preamplifier (1000×gain) and sent to a general purpose computer for spike detection, further filtering, and recording. The local field potentials were digitized at a 2 kHz sampling rate and the action potentials were digitized at a 25 kHz sampling rate. Stimulator headstages were constructed from 4-layer printed circuit boards having surface-mount multiplexers to route stimuli to the appropriate microelectrodes. Surface-mount connectors were used to interface the stimulator headstage with the recording headstage and the microelectrode array. The stimulator headstage was controlled using a custom-built stimulator system and stimulation parameters were adjusted online through the general purpose computer.

Biphasic, rectangular voltage pulses (positive phase first) were applied through the microelectrode array. FIG. 6 shows microelectrode recordings before and after a stimulation pulse at varying voltage levels. As shown here, neurostimulation delivered by a microelectrode array (at the appropriate level of intensity) was effective in evoking action potential activity. These results demonstrate that microelectrode stimulation can reliably result in neural activity. Thus, by maintaining the activity of the neurons at a steady, ongoing rate, it is believed that bursting of the neurons can be prevented, resulting in improved seizure control.

The foregoing description and examples have been set forth merely to illustrate the invention and are not intended to be limiting. Each of the disclosed aspects and embodiments of the present invention may be considered individually or in combination with other aspects, embodiments, and variations of the invention. Modifications of the disclosed embodiments incorporating the spirit and substance of the invention may occur to persons skilled in the art and such modifications are within the scope of the present invention.

What is claimed is:

1. A method for treating a neurologic condition in a mammalian subject, comprising:
providing distributed stimulation to a site in the brain of the subject in an ongoing fashion, wherein the brain site has neurons capable of exhibiting pathologic increases in correlated neural activity.

2. The method of claim 1, wherein the stimulation is electrical stimulation.

3. The method of claim 2, further comprising:
positioning a microelectrode array having a plurality of microelectrodes at the brain site; and
applying a stimulation signal through at least one of the microelectrodes.

4. The method of claim 3, wherein the stimulation signal has a frequency in the range of 0.5-200 Hz and a current in the range of ±0.1-100 μA.

5. The method of claim 3, wherein a stimulation signal is applied through two or more of the microelectrodes, and wherein the stimulation signal through each of the microelectrodes is independently controlled.

6. The method of claim 1, wherein the neurologic condition is epilepsy.

7. The method of claim 6, wherein the distributed stimulation to the brain site is provided for a duration encompassing at least a portion of an interictal period.

8. The method of claim 7, wherein the distributed stimulation to the brain site is provided for a duration encompassing the entire duration of the interictal period.

9. The method of claim 7, wherein the distributed stimulation to the brain site results in the suppression of burst activity at the brain site.

10. The method of claim 7, wherein the distributed stimulation modifies the firing rate of the neurons at the brain site.

11. The method of claim 1, further comprising detecting electrophysiologic signals at the brain site.

12. The method of claim 11, further comprising:
analyzing the detected electrophysiologic signals; and
modifying the stimulation according to a closed-loop feedback algorithm.

13. The method of claim 11, wherein the detecting of electrophysiologic signals is performed simultaneously or continuously with providing the distributed stimulation.

14. The method of claim 11, further comprising extracting a frequency band content from the electrophysiologic signals.

15. The method of claim 14, wherein the content of the frequency band includes single or multi-unit action potentials spikes.

16. The method of claim 15, wherein the frequency band comprises a frequency range of 500-9,000 Hz.

17. The method of claim 14, wherein the content of the frequency band includes local field potentials.

18. The method of claim 17, wherein the frequency band comprises a frequency range of 1-500 Hz.

19. The method of claim 3, further comprising detecting electrophysiologic signals at the brain site through one or more of the microelectrodes.

20. The method of claim 19, wherein the application of a stimulation signal to a microelectrode and the detection of electrophysiologic signals through a microelectrode are performed through the same microelectrode in an alternating fashion.

21. The method of claim 19, further comprising:
analyzing the detected electrophysiologic signals; and
modifying the stimulation signal according to a closed-loop feedback algorithm.

22. The method of claim 21, wherein the closed-loop feedback algorithm uses the detected array-wide firing rate of the neurons throughout the microelectrode array.

23. The method of claim 21, wherein the voltage or the current of the stimulation signal is modified.

24. The method of claim 21, wherein the modification of the stimulation signal is performed in real-time.

25. The method of claim 1, wherein the stimulation is optical stimulation.

26. The method of claim 25, wherein at least some of the neurons at the target site have light-activated ion channels.

27. A system comprising:
a microelectrode array having a plurality of microelectrodes; and
a stimulator subsystem coupled to the microelectrode array;
wherein the stimulator subsystem is programmed to apply a plurality of stimulation signals to the microelectrodes in an ongoing fashion to provide distributed electrical stimulation to a site in the brain of a mammalian subject.

28. The system of claim 27, wherein each of the plurality of electrical signals have a frequency in the range of 0.5-200 Hz and a current in the range of ±0.1-100 μA.

29. The system of claim 28, wherein the array-wide frequency of the distributed electrical stimulation is in the range of 50-200 Hz.

30. The system of claim 27, wherein the stimulator subsystem is adapted to apply current-controlled stimulation.

31. The system of claim 27, wherein the stimulator subsystem is adapted to apply voltage-controlled stimulation.

32. The system of claim 27, further comprising a detector subsystem for detecting electrophysiologic signals from the brain site, and wherein at least one of the microelectrodes is adapted for detecting electrophysiologic signals from the brain site.

33. The system of claim 32, wherein the at least one microelectrode is adapted for detecting single or multi-unit neuronal activity.

34. The system of claim 32, wherein the detector subsystem includes a signal filter for extracting a frequency band content from the detected electrophysiologic signals.

35. The system of claim 34, wherein the frequency band content includes single or multi-unit action potentials spikes.

36. The system of claim 34, wherein the signal filter extracts a frequency band that comprises a frequency range of 300-10,000 Hz.

37. The system of claim 34, wherein the signal filter extracts a frequency band that comprises a frequency range of 0-500 Hz.

38. The system of claim 32, wherein the detector subsystem provides closed-loop feedback to the stimulator subsystem.

39. The system of claim 38, wherein the feedback operates in real-time.

40. The system of claim 38, further comprising a computer having executable instructions for analyzing the detected electrophysiologic signals and modifying the stimulation signals based on a closed-loop feedback algorithm.

41. A computer-readable storage medium having executable instructions for performing the following:
receiving electrophysiologic signals from a microelectrode array;
analyzing the electrophysiologic signals;
using a closed-feedback loop algorithm, modifying the parameters for providing distributed electrical stimulation through the microelectrode array in an ongoing fashion; and
outputting a message containing commands for providing the distributed electrical stimulation.

42. The computer-readable storage medium of claim 41, wherein the step of analyzing the electrophysiologic signals comprises determining the detected firing rate throughout the microelectrode array.

43. The computer-readable storage medium of claim 41, wherein the step of modifying the parameters comprises changing the voltage or current used in the electrical stimulation.

44. The computer-readable storage medium of claim 41, wherein the step of analyzing the electrophysiologic signals comprises determining the power spectrum of the electrophysiologic signals.

45. The computer-readable storage medium of claim 41, further comprising cross-correlating the content of two different frequency bands within the detected electrophysiologic signals.

46. A method for treating a neurologic condition in a mammalian subject, comprising:
positioning a microelectrode array having a plurality of microelectrodes at a site in the brain of the subject having neurons capable of exhibiting pathologic increases in correlated neural activity;
providing distributed electrical stimulation to the brain site by applying a stimulation signal through at least one of the microelectrodes in an ongoing fashion;
detecting electrophysiologic signals at the brain site through at least one of the microelectrodes of the microelectrode array;
analyzing the detected electrophysiologic signals; and
modifying the stimulation signal according to a closed-loop feedback algorithm.
